(19) United States
(12) Patent Application Publication
(10) Pub. No.: US 2005/0079181 A1
(43) Pub. Date: Apr. 14, 2005

(54) Use of immunoglobulins from egg yolk to treat infections caused by parasites both in animals and in humans

(76) Inventors: Jose Andres Morales Garzon, Tehuacan (MX); Eduardo Lucio Decanini, Tehuacan (MX)

Correspondence Address: LOCKE LIDDELL & SAPP LLP;ATTN: SUE COTT, 2200 ROSS AVENUE, SUITE 2200, DALLAS, TX, 75201-6776, US

(21) Appl. No.: 10/803,609

(22) Filed: Mar. 18, 2004

(30) Foreign Application Priority Data
Mar. 18, 2003 (MX) PA/A/2003/003959

(51) Int. Cl. A61K 39/395; C07K 16/20

(52) U.S. Cl. 424/151.1; 530/388.6; 800/4

(57) ABSTRACT

This invention is related to the use of immunoglobulins obtained from the egg yolk of hens hyperimmunized with a mixture of oocysts from several Eimerias species, but can be extended to any human or animal parasites. The immunoglobulins are obtained through the aqueous phase extraction of egg yolk through the use of hydroxypropylmethylcellulose phtalate at a final concentration of 0.05% and sodium azide at a final concentration of 0.001% as preservative. This invention is related also to the administration of said immunoglobulins in powder form in the food at a rate of 0.5 kg per ton of finished food. The administration of immunoglobulins both in liquid as well as in powder form lowers mortality, lesions and counts of coccidian oocysts in treated animals. On the other hand, the use of immunoglobulins produces weight gain in the treated animals.

### FIELD OF THE INVENTION

This invention consists in offering a new treatment and prevention methods for infections caused by parasites in animals (birds, pigs, cattle, and small species) and in humans based on the oral and parenteral administration of immunoglobulins obtained from the egg yolk of hens hyperimmunized with said parasites.

### BACKGROUND OF THE INVENTION

There are two ways of protecting animals against infectious agents: they can exposed to antigens derived from an infectious agent to stimulate a protective immune reaction or they can receive a preformed antibody obtained from an immune subject. The first way is conducted through different types of vaccines: freeze-dried live viruses or bacteria, dead viruses o bacteria in oily emulsions; and recently the creation of cloned and recombinant vaccines. Each of them presents advantages and drawbacks with regard to protection, immune response and protection duration. Besides, in some cases, there are undesirable lesions in the host because of the vaccine virus (Tizard, I. R. 1998)

The second form of protection, also called passive immunity, includes the transmission of antibodies specific against infectious agents in a host.

Traditionally, at research level, the antibodies are mainly obtained in mammals and less frequently in birds. The types of antibodies obtained are monoclonal and polyclonal antibodies in mammals and polyclonal antibodies in birds (Larsson, et al. 1993).

In the case of birds, the chicken is the only species from which antibodies are obtained in a most accessible and highly defined form. The main serum antibody present in the chicken is IgG, even though IgG is transported to the egg in a way similar to the transfer of mammal IgG through the placenta. In the egg, IgG is found in higher concentrations in the yolk, although it is also found in small concentrations in the white; it is even found in larger quantities in the yolk than in the hen serum (Larsson, et al. 1993).

To have an idea of the quantity of antibodies made in the hen, we must take into account that an egg-laying hen produces approximately 5 to 6 eggs per week with a yolk volume of about 15 ml. Thus, in a week, a hen produces antibodies in yolk equivalent to 90-100 ml of serum or 180-200 ml of whole blood. This is to be compared with the 20 ml of whole blood given per week by an immunized rabbit.

Obviously if we use animals such as horses or cows, the quantity of serum and antibodies is larger than in the egg but it is more expensive and more painful for the animals. Among the advantages of the antibodies found in the yolk of hen egg, we can mention the following ones:

1.—They do not fix the complement

2.—They do not bind to the Protein A of Staphilococdus aureus

3.—They do not react with the Rheumatoid Factor

4.—Because of its phylogenetic difference with mammal antibodies, the IgG does not cross react with the mammal antibodies.

5.—Low cost.

Recently, egg yolk antibodies (immunoglobulins) have been employed as tools for diagnostic and therapy (Schmidt, et al. 1989). Thus, taking advantage of its phylogenetic difference with mammal immunoglobulins, the Ig's have presented several advantages when used in immune diagnosis. For example, yolk Ig's have been used to detect several viruses through ELISA, immunodiffusion, immunofluorescence and complement fixing. Because of their low isoelectric point, compared to human Ig, they are employed in electrophoresis assays for the quantification of immunoglobulins in the serum of several animals (Altschuh, D. 1984, Larsson, et al. 1988, Larsson, et al. 1992, Larsson, et al. 1993, Schade, R. 1996). With regard to their therapeutic application, the Ig's have been used as immunotherapy in several scientific fields. For example, the administration of egg yolk immunoglobulins orally has prevented rotavirus infections in mice, bovines, and pigs, among others (Ikemori, et al 1992, Kuroki, et al 1994, Marquardt, et al 1998). Moreover, they have been used as antivenins against viper and scorpions, that can be injected to neutralize the toxins without the risk of anaphylactic reactions commonly caused by antivenins elaborated in horse (Larsson, et al. 1993). A further application has been to prevent caries caused by Streptococcus mutans in humans (Hatta, H. et al 1984).

Objects, Uses and Advantages of the Instant Invention

The object of the instant invention is to offer a prevention and/or treatment method of illnesses caused by protozoans in animals, including human beings, through the oral administration, in aqueous solution or in dry form mixed with food or through parenteral route, of immunoglobulins specifically directed against said parasites (coccidia, fasciola, amebas, isospora, or any other parasite genus) obtained from the egg yolk of hyperimmunized hens.

Another object of the instant invention is to offer weight gain to the animals treated with immunoglobulins specifically directed against birds, pigs or cattle parasites (coccidia, fasciola, amebas, isospora and any other parasite genus).

Moreover, within the scope of the instant invention, the use of egg yolk inmmunoglobulins against parasites is claimed to eliminate or reduce substantially the signology, mortality and transmission in the treated animals.

Finally, the invention is focused on a process to prepare a product based on immunoglobulins specifically directed against parasites of animals or human beings, obtained from the egg yolk of hyperimmunized hens.

This invention, in the case of animals, the quantity of oocysts of protozoans in the digestive tract diminishes, and the productive parameters of the animals improve. The immunoglobulins obtained in powder are administered orally mixed with the food.

### DETAILED DESCRIPTION OF THE INVENTION

The detailed characteristics of this novel invention are clearly shown in the following description and in the attached figures.

The instant invention is based on the fact that the immunoglobulins extracted from the aqueous phase of egg yolk offer protection against viral and bacterial illnesses.

Hen Immunization Program

To obtain the immunoglobulins (Igs) specifically directed against animal or human parasites, it is necessary to have a vaccination schedule in a flock of SPF (Specific Pathogens Free) birds.

The vaccination schedule can include the administration, orally, subcutaneously or through any other way of the parasites, alive or dead, through any chemical method. The parasites are included in an oily or semi-oily vehicle or in a vehicle of any other type in such a way as to ensure an immune response in the hen. The recommended dose is 0.5 ml in laying hens in the growing stage at 8, 12 and 16 weeks of age.

Obtaining Immunoglobulins from Powder Yolk

In short, the process was as follows. The yolk is separated from the white and diluted 1:2 with a 0.005% sodium azide solution and is thoroughly mixed with the help of a stirrer. Once the yolk is diluted, it is dried through the Spry Dried method. The quality control tests include:

1.—Sterility test (to check if the product is free from contamination by bacteria, fungi and yeast according to the Code of Federal Regulations 9 of the United States of America.

2.—Antibodies quantification. ELISA techniques or any other method are used to detect the antibodies for each species.

Hereinafter tests are presented as non-limiting examples, showing the use of immunoglobulins against Coccidia in broiler chicken, object of the instant invention.

### EXAMPLE 1

The experimental design was conducted in the following way: 5 groups of broiler chicken of 3 weeks of age were formed. The first group received 0.5 ml immunoglobulins in a solution administered orally on the day the groups were formed and a second time 8 days later. Group 2 received 1 ml, group 3 received 2 ml, group 4 received 4 ml and group 5 was the control group without immunoglobulins treatment. All the groups were fed with food without anticoccidian and ten days after the groups were formed, approximately wend the groups where 31 days of age all the groups were challenged with 200,000 oocysts of E. tenella. The animals were sacrificed 7 days after to determine lesions according to the Johnson and Reid's scale. The results are given in Table 1. It can be observed that the immunoglobulins treatment against coccidia lowers mortality and caeca weight even at a 0.5 ml concentration treatment with only two doses at 7-day interval between the first and the second.

### EXAMPLE 2

Two groups of broiler birds were formed: one group received 1 ml of immunoglobulins in daily doses through drinking water during two weeks. The other group, the control group, did not receive any treatment. After two weeks of treatment, both groups were challenged with 150,000 sporulated oocysts of E. Tenella.

All the animals were sacrificed 7 days later and the caeca lesions were qualified according to Johnson and Reid's scale. Mortality was also recorded. Results are given in Table 2. As can be seen, there was a 28.3% lowering of mortality in the treated group compared to the control group. In the same way, a 53.7% lowering of the caeca weight can be observed in the treated group compared to the control group without treatment.

### EXAMPLE 3

Three groups of one-week old broiler birds were formed. The first group received 2 ml of immunoglobulins against coccidia (Supracox) through drinking water on a daily basis during 14 days and they were fed with food without anticoccidian. The second group was fed with an anticoccidian (maduramicine ammonium or clopidol), without administration of immunoglobulins and the last group was not treated with Immunoglobulins and the food did not contain anticoccidian, said group being a negative control group. After the treatment period with immunoglobulins, all groups were challenged with 200,000 oocysts of a mixture of Eimerias acervulina, E. brunetti, E. maxima and E. tenella. The parameters to evaluate were weight gain, oocysts counts in caecal and intestinal contents and conversion index. Results are presented in Table 3, in which a 68.5% weight gain can be observed in the group treated with immunoglobulins compared to the control group. With regard to oocysts recovery, in the treated group there was no recovery while in the group that received food with anticcoccidians, the count was 192,000 and 288,000 oocysts in the control group, without treatment. In the same way a better conversion index was observed with the group treated with immunoglobulins compared to the group without treatment.

### TABLES

TABLE 1
Results with the use of immunoglobulins supplied in drinking water.

| | Immunoglobulins | | | |
|---|---|---|---|---|
| | Volume | % | Caeca | % |
| Group | MI | Mortality | weight | Protection |
| 1 | 0.5 | 12.5 | 8.8 | 64.0 |
| 2 | 1.0 | 37.5 | 24.0 | 0.5 |
| 3 | 2.0 | 37.5 | 14.0 | 42.0 |
| 4 | 4.0 | 25.0 | 11.6 | 52.0 |
| 5 | 0.0 | 25.0 | 24.1 | 0.0 |

TABLE 2
Results obtained with the use of 1 ml of
immunoglobulin against coccidia during 14 days orally in
drinking water.

| | | Mortality | Caeca weight |
|---|---|---|---|
| Group | Immunoglobulin volume (ml) | % | (g) |
| Treated | 1 ml/drinking water/14 days | 55 | 16.2 |
| Controls | Without treatment with Igs | 83.3 | 35.0 |

TABLE 3
Results of a comparative test using immunoglobulins
against coccidia in drinking water and food with
anticoccidian.

| | | Final | Oocysts in | Occysts in | |
|---|---|---|---|---|---|
| | | weight | caeca | intestinal | Conversión |
| Group | Treatment | gain (g) | contents | contents | Index |
| 1 | 1 ml Igs/14 | 337.0 | 0.0 | 0.0 | 2.33 |
| | days | | | | |
| 2 | Food with | 234.0 | 288,000 | 192,000 | 3.22 |
| | anticoccidian | | | | |
| 3 | Food without | 200 | 576,000 | 288,000 | 3.90 |
| | anticoccidian | | | | |

1. The use of immunoglobulins for the treatment of infections caused by protozoans in animals and human beings, obtained through the exhaustive vaccination of light SPF-type egg-laying birds with an oocysts suspension of dead or live parasites administered orally or parenterally.

2. The immunoglobulins of claim 1 obtained through the aqueous phase extraction from egg yolk.

3. The immunoglobulins of claim 1 obtained through the use of 0.001% sodium azide and 5% hydroxypropylmethylcellulose phtalate.

4. The immunoglobulins of claim 1 that can also be dehydrated by a Spray Dried method to be administered to food at a rate of 0.5 kg per ton of food.

5. The immunoglobulins of claim 1 lower the rate of mortality caused by a challenge with one or several Eimeria species.

6. The immunoglobulins of claim 1 lower the lesions caused by the Eimeria species where aqueous administration is made at a daily rate of 2 ml per bird.
